(19) United States
(12) Patent Application Publication
(10) Pub. No.: US 2024/0122859 A1
(43) Pub. Date: Apr. 18, 2024

(54) EXTENDED RELEASE MULTIPLE UNIT PELLET SYSTEM COMPOSITION AND ITS PROCESS FOR THE PREPARATION

(71) Applicants: LYRUS LIFE SCIENCES PVT. LTD., Bangalore (IN); NOKHA TRADING LLP, Bangalore (IN)

(72) Inventors: Chandanmal Pukhraj Bothra, Bangalore (IN); Hemanth Kumar Bothra, Bangalore (IN); Elayaraja Natarajan, Bangalore (IN); Chitra Varma, Bangalore (IN); Bharat Ghanshyam Rughwani, Bangalore (IN); Vaibhav Prakash Thorat, Bangalore (IN)

(21) Appl. No.: 18/276,864

(22) PCT Filed: Feb. 12, 2022
(86) PCT No.: PCT/IB2022/051261

§ 371 Date: Aug. 10, 2023

(30) Foreign Application Priority Data
Feb. 12, 2021 (IN) 202141005996

(51) Int. Cl. A61K 9/20; A61K 9/28

(52) U.S. Cl. CPC A61K 9/2081; A61K 9/2095; A61K 9/2813; A61K 9/282; A61K 9/2866

(57) ABSTRACT

The present invention relates to the novel extended release multiple unit pellet system composition and process for the preparation. The present invention specifically relates to novel extended release multiple unit pellet system composition comprising active pharmaceutical ingredient and pharmaceutically acceptable excipients. The present invention also relates to novel dispersible extended release multiple unit pellet system composition comprising active pharmaceutical ingredient and pharmaceutically acceptable excipients, wherein the active pharmaceutical ingredient is present in the core spheres. The present invention more specifically relates to novel process for the preparation of extended release multiple unit pellet system composition using dual seal coating technology.

### FIELD OF INVENTION

The present invention relates to the novel extended release multiple unit pellet system composition and process for the preparation.

The present invention specifically relates to novel extended release multiple unit pellet system composition comprising active pharmaceutical ingredient and pharmaceutically acceptable excipients.

The present invention also relates to novel dispersible extended release multiple unit pellet system composition comprising active pharmaceutical ingredient and pharmaceutically acceptable excipients, wherein the active pharmaceutical ingredient is present in the core spheres.

The present invention also relates to novel dispersible extended release multiple unit pellet system composition comprising active pharmaceutical ingredient and pharmaceutically acceptable excipients, wherein the active pharmaceutical ingredient and excipients are present in the core spheres.

The present invention more specifically relates to novel process for the preparation of extended release multiple unit pellet system composition using dual seal coating technology.

The present invention also relates to novel process for the preparation of extended release multiple unit pellet system composition using dual seal coating technology which yields product with low level of Nitrosamine compared to conventional techniques.

The present invention also relates to novel dispersible extended release multiple unit pellet system which can be dispersed in water or any suitable medium before administration.

### BACKGROUND OF INVENTION

Multiple unit pellet systems (MUPS) are seen as superior in terms of intra- and inter-individual variability of in vivo drug absorption (Lehmann K, Petereit H U, Dreher D 1993, Pharm Ind 55:940-947) in combination with oral modified release products. These pellets can be filled into hard capsules or be compressed together with suitable fillers and binders into disintegrating pellet-containing tablets. The main focus of the prior art is to retain the modified release properties of the single units, whether those are controlled via a film coat or the embedding of the active ingredient into a polymeric matrix (Abdul S, Chandewar A V, Jaiswal S B 2010, J Control Release 147:2-16).

In order to achieve an optimal product the following requirements should be fulfilled (Wagner K G, Krumme M, Schmidt P C 1999. Investigation of the pellet-distribution in single tablets via image analysis. Eur J Pharm Biopharm 47:79-85):
The filler matrix needs to absorb the main fraction of the compression energy and simultaneously provide an acceptable values of tensile strength and hardness.
The fraction of pellets within the formulation should not exceed 60-70% (w/w).
Where coated pellets should be processed into tablets, the polymer coating needs to exhibit a suitable elasticity to cope with some deformation during tableting without rupture.

The purpose of the present invention is to provide a novel extended release multiple unit pellet system composition and its process for the preparation. It is another purpose of the invention to provide a process that can be used on an industrial scale for preparing extended release MUPS containing pharmaceutically active agent and excipients. The purpose of developing MUPS is to provide the drug release over a period of time and to address the needs of the 40% of adults who cannot or will not swallow solid medications. The Dispersible MUPS can be taken as whole tablet or dispersed in water or apple sauce at the time of administration which is very useful for geriatrics and patients who cannot swallow.

### Objective of Invention

The main objective of the present invention is to provide novel extended release multiple unit pellet system composition and process for the preparation.

Another objective of the present invention is to provide novel extended release multiple unit pellet system composition comprising active pharmaceutical ingredient and pharmaceutically acceptable excipients.

Another objective of the present invention is to provide novel extended release multiple unit pellet system composition comprising active pharmaceutical ingredient, release controlling polymers, diluents, coating polymers, disintegrants, plasticizers, binders, glidants, anti-tacking agents or anti-adherents and lubricants.

Another objective of the present invention is to provide dispersible multiple unit pellet system composition which can be dispersed in water or apple sauce at the time of administration and is very useful for geriatric patients or patients who cannot swallow.

Still another objective of the present invention is to provide novel process for the preparation of extended release multiple unit pellet system composition using dual seal coating technology comprising the steps of extrusion spheronization, seal coating, functional coating, seal coating, adding extragranular excipients and compression or filling in sachets or capsules.

### SUMMARY OF INVENTION

Accordingly, the present invention provides a novel extended release multiple unit pellet system composition and process for the preparation.

One embodiment of the present invention provides a novel extended release multiple unit pellet system composition comprising active pharmaceutical ingredient and pharmaceutically acceptable excipients.

Another embodiment of the present invention provides a novel extended release multiple unit pellet system composition comprising active pharmaceutical ingredient, release controlling polymers, diluents, coating polymers, disintegrants, plasticizers, binders, glidants, anti-tacking agents or anti-adherents and lubricants.

Another embodiment of the present invention relates to a novel dispersible extended release multiple unit pellet system composition comprising active pharmaceutical ingredient and pharmaceutically acceptable excipients wherein the active pharmaceutical ingredient is present in the core spheres.

Another embodiment of the present invention relates to a novel dispersible extended release multiple unit pellet system composition comprising active pharmaceutical ingredient and pharmaceutically acceptable excipients wherein the active pharmaceutical ingredient and excipients are present in the core spheres.

Another embodiment of the present invention provides a dispersible multiple unit pellet system composition can be dispersed in water or apple sauce at the time of administration which is very useful for geriatric patients and patients who cannot swallow.

Another embodiment of the present invention provides a novel process for the preparation of extended release multiple unit pellet system composition by using dual seal coating technology comprising the steps of extrusion spheronization, seal coating, functional coating, seal coating, adding extragranular excipients and compression or filling in sachets or capsules.

Another embodiment of the present invention provides novel process for the preparation of extended release multiple unit pellet system composition by using dual seal coating technology which yields product with low level of Nitrosamine compared to conventional techniques.

Another embodiment of the present invention provides a novel extended release multiple unit pellet system composition is prepared by coating active pharmaceutical ingredient core spheres with seal coating of 5% buildup using coating polymer selected from polyethylene glycol, hypromellose, povidone, Eudragit and Opadry and purified water, functional coating of 50% buildup using ethyl cellulose and/or hypromellose, triethyl citrate, talc, isopropyl alcohol and purified water, second seal coating of 5% buildup over functional coated pellets using coating polymer selected from polyethylene glycol, hypromellose, povidone, Eudragit and Opadry and purified water, extragranular portion containing microcrystalline cellulose, copovidone, colloidal silicon dioxide and sodium stearyl fumarate or magnesium stearate for lubricating and compressing to get multiple unit pellet system.

Another embodiment of the present invention provides a novel extended release multiple unit pellet system composition is prepared by coating active pharmaceutical ingredient core spheres with seal coating of 5% buildup using coating polymer selected from polyethylene glycol, hypromellose, povidone, Eudragit and Opadry and purified water, functional coating of 50% buildup using ethyl cellulose dispersion with or without hypromellose and purified water, second seal coating of 5% buildup over functional coated pellets using coating polymer selected from polyethylene glycol, hypromellose, povidone, Eudragit and Opadry and purified water, extragranular portion containing microcrystalline cellulose, copovidone, colloidal silicon dioxide and sodium stearyl fumarate or magnesium stearate for lubricating and compressing to get multiple unit pellet system.

Yet another embodiment of the present invention provides a composition of extended release multiple unit pellet system comprising:
(a) core spheres containing active pharmaceutical ingredient and optionally excipients,
(b) seal coating over the core containing coating polymer and one or more solvents,
(c) functional coating over seal coated pellets containing release controlling polymers and other excipients, and
(d) second seal coating over the functional coated pellets containing coating polymer and one or more solvents.

Yet another embodiment of the present invention provides a composition of extended release multiple unit pellet system comprising:
(a) core spheres containing active pharmaceutical ingredient and optionally excipients,
(b) seal coating over the core containing coating polymer and one or more solvents,
(c) functional coating over seal coated pellets containing release controlling polymers and other excipients,
(d) second seal coating over the functional coated pellets containing coating polymers and one or more solvents, and
(e) extra-granular portion containing disintegrant, binder, glidant and lubricant.

Yet another embodiment of the present invention provides a composition of extended release multiple unit pellet system comprising:
(a) core spheres containing active pharmaceutical ingredient, diluent and binder,
(b) seal coating over the core containing coating polymer and one or more solvents,
(c) functional coating over seal coated pellets containing release controlling polymers, plasticizer, anti-adherent and one or more solvents,
(d) second seal coating over the functional coated pellets containing coating polymers and one or more solvents, and
(e) extra-granular portion containing disintegrant, binder, glidant and lubricant.

Yet another embodiment of the present invention provides a composition of extended release multiple unit pellet system comprising:
(a) core containing Metformin hydrochloride, microcrystalline cellulose and binder selected from povidone, hydroxy propyl cellulose and copovidone,
(b) seal coating over the Metformin hydrochloride core containing coating polymer selected from polyethylene glycol, hypromellose, povidone, Eudragit and Opadry and purified water,
(c) functional coating over seal coated pellets containing release controlling polymer selected from one or more of ethyl cellulose, hypromellose, aqueous ethylcellulose dispersion and other excipients,
(d) second seal coating over the functional coated pellets containing coating polymer selected from polyethylene glycol, hypromellose, povidone, Eudragit and Opadry and purified water,
(e) extra-granular portion containing microcrystalline cellulose, copovidone, colloidal silicon dioxide and lubricant selected from magnesium stearate and sodium stearyl fumarate.

Yet another embodiment of the present invention provides a composition of extended release multiple unit pellet system comprising:
(a) core containing Metoprolol succinate, microcrystalline cellulose and binder selected from povidone, hydroxy propyl cellulose and copovidone,
(b) seal coating over the Metoprolol succinate core containing coating polymer selected from polyethylene glycol, hypromellose, povidone, Eudragit and Opadry and purified water,
(c) functional coating over seal coated pellets containing release controlling polymer selected from one or more of ethyl cellulose, hypromellose, aqueous ethylcellulose dispersion and purified water,
(d) second seal coating over the functional coated pellets containing coating polymer selected from polyethylene glycol, hypromellose, povidone, Eudragit and Opadry and purified water,
(e) extra-granular portion containing microcrystalline cellulose, copovidone, colloidal silicon dioxide and lubricant selected from magnesium stearate and sodium stearyl fumarate.

Yet another embodiment of the present invention provides a composition of extended release multiple unit pellet system comprising:
(a) core containing Venlafaxine hydrochloride, microcrystalline cellulose and povidone,
(b) seal coating over the Venlafaxine hydrochloride core containing polyethylene glycol and purified water,
(c) functional coating over seal coated pellets containing ethyl cellulose, hypromellose, triethyl citrate, talc, Isopropyl alcohol and purified water,
(d) second seal coating over the functional coated pellets containing polyethylene glycol and purified water, and
(e) extra-granular portion containing microcrystalline cellulose, copovidone, colloidal silicon dioxide and magnesium stearate.

In yet another embodiment of the present invention provides an extended release multiple unit pellet system composition, wherein tablet obtained is optionally coated with Opadry ready to use film coating is selected from and not limited to Opadry II® (Y-30-13579-A), Opadry Clear® (YS-2-19114-A), Opadry II white, Opadry II brown, Opadry 11 orange, or Opadry II yellow.

In yet another embodiment, the present invention provides a novel process for the preparation of extended release multiple unit pellet system composition comprising steps of:
(a) extruding and spheronizing active ingredient and optionally excipients,
(b) mixing seal coating polymer with water or any other solvent and coating over extruded spheres obtained in step (a) to form seal coated pellets,
(c) adding and mixing release controlling polymers, in water or any other solvent under vigorous stirring and coating over seal coated pellets obtained in step (b) to form functional coated pellets,
(d) mixing seal coating polymer with water or any other solvent and coating over pellets obtained in step (c) to form final coated pellets,
(e) blending the obtained final coated pellets obtained in step (d) with diluent, disintegrant, binder, glidant, lubricant, and
(f) compressing the blended pellets obtained in step (e) into tablet or filling the blended pellets obtained in step (e) into capsules or sachets.

In yet another embodiment, the present invention provides a novel process for the preparation of extended release multiple unit pellet system composition comprising steps of:
(a) extruding and spheronizing active ingredient, diluent and binder to form extruded spheres,
(b) mixing seal coating polymer with water or any other solvent and coating over extruded spheres obtained in step (a) to form seal coated pellets,
(c) adding and mixing release controlling polymers, anti-adherent, plasticizer in water or any other solvent under vigorous stirring and coating over seal coated pellets obtained in step (b) to form functional coated pellets,
(d) mixing seal coating polymer with water or any other solvent and coating over pellets obtained in step (c) to form final coated pellets,
(e) blending the obtained final coated pellets obtained in step (d) with diluent, disintegrant, binder, glidant, lubricant, and
(f) compressing the blended pellets obtained in step (e) into tablet or filling the blended pellets obtained in step (e) into capsules or sachets.

In yet another embodiment, the present invention provides novel process for the preparation of extended release multiple unit pellet system composition comprising steps of:
(a) extruding and spheronizing Metformin hydrochloride, microcrystalline cellulose and binder selected from povidone, hydroxy propyl cellulose and copovidone to form extruded spheres,
(b) mixing coating polymer selected from polyethylene glycol, hypromellose, povidone, Eudragit and Opadry with water and coating over extruded spheres obtained in step (a) to form seal coated pellets,
(c) adding and mixing release controlling polymer selected from one or more of ethyl cellulose, hypromellose, aqueous ethylcellulose dispersion in water under vigorous stirring and coating over seal coated pellets obtained in step (b) to form functional coated pellets,
(d) mixing coating polymer selected from polyethylene glycol, hypromellose, povidone, Eudragit and Opadry with water and coating over functional coated pellets obtained in step (c) to form final coated pellets,
(e) blending the coated pellets obtained in step (d) with microcrystalline cellulose, copovidone, colloidal silicon dioxide and lubricating with magnesium stearate or sodium stearyl fumarate, and
(f) compressing the mixed pellets obtained in step (e) into tablet or filling the blended pellets obtained in step (e) into capsules or sachets.

In yet another embodiment, the present invention provides novel process for the preparation of extended release multiple unit pellet system composition comprising steps of:
(a) extruding and spheronizing Metoprolol succinate, microcrystalline cellulose and binder selected from povidone, hydroxy propyl cellulose and copovidone,
(b) mixing coating polymer selected from polyethylene glycol, hypromellose, povidone, Eudragit and Opadry with water and coating over extruded spheres obtained in step (a) to form seal coated pellets,
(c) adding and mixing release controlling polymer selected from one or more of ethyl cellulose, hypromellose, aqueous ethylcellulose dispersion in water under vigorous stirring and coating over seal coated pellets obtained in step (b) to form functional coated pellets,
(d) mixing coating polymer selected from polyethylene glycol, hypromellose, povidone, Eudragit and Opadry with water and coating over functional coated pellets obtained in step (c) to form final coated pellets,
(e) blending the coated pellets obtained in step (d) with microcrystalline cellulose, copovidone, colloidal silicon dioxide and lubricating with magnesium stearate or sodium stearyl fumarate, and
(f) compressing the mixed pellets obtained in step (e) into tablet or filling the blended pellets obtained in step (e) into capsules or sachets.

In yet another embodiment, the present invention provides novel process for the preparation of extended release multiple unit pellet system composition comprising steps of:
(a) extruding and spheronizing Venlafaxine hydrochloride, microcrystalline cellulose and povidone to form extruded spheres,
(b) mixing polyethylene glycol with water and coating over extruded spheres obtained in step (a) to form seal coated pellets,
(c) adding and mixing ethyl cellulose and hypromellose, triethyl citrate in water under vigorous stirring and coating over seal coated pellets obtained in step (b) to form functional coated pellets,
(d) mixing polyethylene glycol with water and coating over functional coated pellets obtained in step (c) to form final coated pellets,
(e) blending the coated pellets obtained in step (d) with microcrystalline cellulose, copovidone, colloidal silicon dioxide and lubricating with magnesium stearate, and
(f) compressing the mixed pellets obtained in step (e) into tablet or filling the blended pellets obtained in step (e) into capsules or sachets.

### DETAILED DESCRIPTION OF THE INVENTION

The term “comprising”, which is synonymous with “including”, “containing”, or “characterized by” here is defined as being inclusive or open-ended, and does not exclude additional, unrecited elements or method steps, unless the context clearly requires otherwise.

The present invention relates to the novel extended release multiple unit pellet system composition and process for the preparation.

The extended release multiple unit pellet system prepared using the multiple unit pellet system composition of the present invention has Nitrosamines levels lower than those products prepared using conventional methods.

The present invention specifically relates to novel extended release multiple unit pellet system composition comprising active pharmaceutical ingredient and pharmaceutically acceptable excipients.

The “multiple unit pellet system” (MUPS) consists of extremely small extended release-coated granules (pellets) of the active pharmaceutical agent. MUPS system is of benefit for those patients requiring nasogastric tube feeding and those with difficulty swallowing (dysphagia) because the tablets can be mixed with water ahead of time, releasing the granules into a slurry form, which is easier to pass down the feeding tube or to swallow than the pill.

Core as used herein can be prepared by extruding processes include hot melt extrusion, melt granulation, extrusion spheronization, direct pelletization, spray drying and spray coagulation granulation, solvent evaporation, fluid bed granulation, spray drying. The advantage of this process is that the process time for production of core spheroids is significantly less than the conventional drug layering approach used in MUPS formulations.

Another embodiment of the present invention provides dispersible multiple unit pellet system composition which can be dispersed in water or apple sauce at the time of administration and is very useful for geriatric patients or patients who cannot swallow.

Another embodiment of the present invention provides novel extended release multiple unit pellet system composition comprising active pharmaceutical ingredient, release controlling polymers, diluents, coating polymers, disintegrants, plasticizers, binders, glidants, anti-tacking agents or anti-adherents and lubricants.

In still yet another embodiment of the present invention, the pharmaceutical composition also relates to a wide variety of active pharmaceutical ingredients suitable for use in controlled release formulations. Representative active pharmaceutical ingredient's may include antibacterial, antacids, analgesic and anti-inflammatory agents, anti-arrhythmic agents, antiprotozoal agents, anti-coagulants, antidepressants, anti-diabetic agents, anti-epileptic agents, antifungal agents, antihistamines, anti-hypertensive agents, anti-muscarinic agents, antineoplastic agents, antimetabolites, anti-migraine agents, anti-parkinsonian agents, antipsychotic, hypnotic and sedating agents, anti-stroke agents, antitussive, antivirals, cardiac inotropic agents, corticosteroids, disinfectants, diuretics, enzymes, essential oils, gastro-intestinal agents, haemostatics, lipid regulating agents, local anaesthetics, opioid analgesics, parasympathomimetics and anti-dementia drugs, peptides and proteins, sex hormones, stimulating agents, vasodilators or mixtures thereof.

According to an embodiment of the present invention, a preferred active pharmaceutical ingredient is a high soluble high dose active pharmaceutical ingredient or derivative thereof. Preferably, the active pharmaceutical ingredient is a high soluble high dose active pharmaceutical ingredient selected from the group consisting of Acebutolol hydrochloride, Amantadine hydrochloride, Aminocaproic acid, Aminophylline, Amodiaquine hydrochloride, Ascorbic acid, Carbenoxolone sodium, Cefuroxime sodium, Chloroquine phosphate, Chloroquine sulphate, Chlorpromazine hydrochloride, Ciprofloxacin hydrochloride, Cloxacillin sodium, Cycloserine, Diltiazem hydrochloride, Diethyl carbamazine citrate, Doxycycline hydrochloride, Ethosuximide, Ferrous gluconate, Isoniazid, Levamisole hydrochloride, Lincomycin hydrochloride, Mebeverine hydrochloride, Mepyramine maleate, Metformin hydrochloride, Metoprolol tartrate, Metoprolol succinate, Nicotinamide, Nicotinic acid, Oxprenolol hydrochloride, Oxytetracycline hydrochloride, Penicillamine, Pentobarbitone sodium, Phenoxy Methyl Penicillin K, Phenyloin sodium, piperazine adipate, Procainamide hydrochloride, Pseudoephedrine hydrochloride, Quinalbarbitone sodium, Quinine bisulphate, Ranitidine hydrochloride, Sodium amino salicylate, Sodium fusidate, Sodium valproate, Streptomycin sulphate, Tetracycline hydrochloride, Troxidone, Potassium chloride, Venlafaxine hydrochloride, Verapamil hydrochloride and the like and their pharmaceutically acceptable salts, ester and hydrates.

In still yet another embodiment of the present invention, a preferred active pharmaceutical ingredient is a low soluble high dose active pharmaceutical ingredient or derivative thereof. preferably, the active pharmaceutical ingredient is a low soluble high dose active pharmaceutical ingredient selected from the group consisting of Acetazolamide, Allopurinol, Atenolol, Carbamazepine, Cefadroxil, Cephalexin, Chloramphenicol, Cefuroxime Axetil, Chlorthalidone, Cimetidine, Clarithromycin, Clofazemine, Curcuminoids and Non-curcuminoids, Dapsone, Diclofenac sodium, Diiodohydroxy quinolone, Diloxanide furoate, Disulfiram, Erythromycin, Erythromycin estolate, Erythromycin stearate, Ethacrynic acid, Ethionamide, Ethopropazine hydrochloride, Ferrous fumarate, Fluconazole, Flurbiprofen, Furazolidone, Griseofulvin, Hydrochlorthiazide, Ibuprofen, Ketoconazole, Ketoprofen, Labetalol hydrochloride, Levodopa, Linezolid, Lithium carbonate, Magaldrate, Mebendazole, Mefenamic acid, Megestrol acetate, Mercaptopurine, Nalidixic acid, Niclosamide, Nitrofurantoin, Norfloxacin, Oxyphenbutazone, Paracetamol, Phenindione, Phenobarbitone, Phenylbutazone, Phenylsulphathiazole, Piperazine phosphate, Proguanil hydrochloride, Promethazine theoclate, Propylthiouracil, Posaconazole, Quinidine sulphate, Quinine sulphate, Quinidochlor, Rifampicin, Spironolactone, Succinylsulphathiazole, Sulphadiazine, Sulphadimethoxine, Sulphadimidine, Sulphafurazole, Sulphaphenazole, Thiabendazole, Tinidazole, Tolbutamide, Triamterene, Sulphamethoxazole and the like and their pharmaceutically acceptable salts, ester and hydrates.

The concentration of active pharmaceutical ingredient used in the extended release multiple unit pellet system is from 10% to 80% (w/w). Preferably used concentration of active ingredient is 20% to 70% (w/w) of the total weight of the composition.

Coating polymer used in the composition of the present invention includes, but not limited to sugars, zein, celluloses, hydroxypropyl celluloses, hydroxypropyl methylcelluloses, hydroxyethyl celluloses, polyvinyl alcohols, polyethylene glycols, poloxamers (e.g., Pluronic™ products), methacryclic acid polymers, poly vinyl acetate, ethylcelluloses, gelatins, polyarginines, polyglycines, polyvinylpyrrolidones (povidone), vinyl acetate copolymers, Opadry coating mixtures and any mixtures thereof. Preferably used coating polymer is polyethylene glycols, hydroxypropyl methylcelluloses, povidone and methacryclic acid polymers.

The concentration of coating polymer used in the extended release multiple unit pellet system is from 1% to 25% (w/w). Preferably used concentration of coating polymer is from 3% to 20% (w/w) of the total weight of the composition.

Release controlling polymers used in the composition of the present invention includes, but not limited to ethyl cellulose, cellulose acetate, cellulose acetate butyrate, ethylene vinylacetate copolymer, polyvidone acetate, polyvinyl acetate, ethyl acrylate/methyl methacrylate copolymer, ammonia methacrylate copolymer, methylcellulose, hydroxy propyl cellulose, hypromellose or combinations thereof. Preferably used release controlling polymers are ethyl cellulose and hypromellose or combination thereof.

The concentration of release controlling polymers used in the extended release multiple unit pellet system is from 5% to 50% (w/w). Preferably used concentration of release controlling polymers in combination is from 10% to 40% (w/w).

Diluent used in the composition of the present invention includes, but not limited to mannitol powder, spray dried mannitol, microcrystalline cellulose, lactose, dicalcium phosphate, tricalcium phosphate, starch, pregelatinized starch, compressible sugars, silicified microcrystalline cellulose, silicon dioxide, and calcium carbonate. Preferably used diluent is microcrystalline cellulose.

The concentration of diluent used in the extended release multiple unit pellet system is from 5% to 60% (w/w). Preferably used concentration of diluent is 5% to 50% (w/w) of the total weight of the composition.

Disintegrant used in the composition of the present invention includes, but not limited to croscarmellose sodium, microcrystalline cellulose, carboxymethyl cellulose sodium, crospovidone, polacrilin potassium and sodium starch glycolate.

The concentration of disintegrant used in the extended release multiple unit pellet system is from 1% to 35% (w/w). Preferably used concentration of disintegrant is from 3% to 30% (w/w) of the total weight of the composition.

Plasticizer used in the composition of the present invention includes, but not limited to polyethylene glycol, triethyl citrate, tributyl citrate, glycerin, dibutyl sebacate, triacetin, glyceryl tricaprylate/caprate, medium chain triglyceride, oleic acid and diethylphthalate. Preferably used plasticizers are triethyl citrate.

The concentration of plasticizer used in the extended release multiple unit pellet system is from 1% to 15% (w/w). Preferably used concentration of plasticizer is from 1% to 10% (w/w) of the total weight of the composition.

Binder used in the composition of the present invention includes, but not limited to methylcellulose, hydroxypropyl cellulose, hydroxypropyl methylcellulo se, povidone, copovidone, gelatin, gum arabic, ethyl cellulose, polyvinyl alcohol, starch, pregelatinized starch, agar, tragacanth or sodium alginate. Preferably used binder is povidone or hydroxypropyl cellulose and preferably used extra-granular binder is copovidone.

The concentration of binder used in the extended release multiple unit pellet system is from 0.1% to 10% (w/w). Preferably used concentration of binder is from 0.5% to 10% (w/w) of the total weight of the composition.

Glidant used in the composition of the present invention includes, but not limited to colloidal silicon dioxide, magnesium trisilicate, powdered cellulose, talc, tribasic calcium phosphate and the like. Preferably used glidant is colloidal silicon dioxide.

The concentration of glidant used in the extended release multiple unit pellet system is from 0.1% to 10% (w/w). Preferably used concentration of glidant is from 0.5% to 5% (w/w) of the total weight of the composition.

Anti-adherent or anti-tacking agent used in the composition of the present invention includes, but not limited to magnesium stearate, talc, calcium stearate, glyceryl behenate, Polyethylene glycols, hydrogenated vegetable oil, mineral oil, stearic acid. Preferably used anti-tacking agent is talc.

The concentration of anti-tacking or anti-adherent used in the extended release multiple unit pellet system is from 0.1% to 10% (w/w). Preferably used concentration of anti-tacking agent is from 0.5% to 5% (w/w) of the total weight of the composition.

Lubricant used in the composition of the present invention includes, but not limited to magnesium stearate, stearic acid, sodium stearyl fumarate, calcium stearate, hydrogenated vegetable oil, mineral oil, polyethylene glycol, polyethylene glycol 4000-6000, talc, and glyceryl behenate. Preferably used concentration of lubricant is magnesium stearate.

The concentration of lubricant used in the extended release multiple unit pellet system is from 0.1% to 10% (w/w). Preferably used concentration of lubricant is from 0.5% to 5% (w/w) of the total weight of the composition.

Solvents used in the composition of the present invention includes, but not limited to isopropyl alcohol, acetone, methanol, ethanol, water and mixtures thereof.

The present invention is to provide novel process for the preparation of extended release multiple unit pellet system composition using dual seal coating technology comprising the steps of extrusion spheronization, seal coating, functional coating, seal coating, adding extragranular excipients and compressing into tablets.

The present invention is to provide novel process for the preparation of extended release multiple unit pellet system composition using dual seal coating technology comprising the steps of extrusion spheronization, seal coating, functional coating, seal coating, adding extragranular excipients and compressing into tablets that can be re-dispersed in water before administration.

The present invention is to provide novel process for the preparation of extended release multiple unit pellet system composition using dual seal coating technology comprising the steps of extrusion spheronization, seal coating, functional coating, seal coating, adding extragranular excipients and filling into capsules or sachets.

The present invention is to provide extended release pellets which are prepared by coating active pharmaceutical ingredient spheres with seal coating of approximately 5% buildup using PEG 4000 and purified water, functional coating of approximately 50% buildup using ethyl cellulose, hypromellose, triethyl citrate, talc, isopropyl alcohol and purified water, seal coating of approximately 5% buildup over functional coated pellets using PEG 4000 and purified water. Obtained pellets were blended with extragranular portion containing microcrystalline cellulose, copovidone, colloidal silicon dioxide, magnesium stearate for lubrication and compressed to get multiple unit pellet system.

The present invention is to provide extended release pellets which are prepared by coating active pharmaceutical ingredient spheres with seal coating of approximately 5% buildup using Opadry clear, hypromellose or povidone and purified water, functional coating of approximately 50% buildup using ethyl cellulose, hypromellose, triethyl citrate, talc, isopropyl alcohol and purified water, seal coating of approximately 5% buildup over functional coated pellets using Opadry clear, hypromellose or povidone and purified water. Obtained pellets were blended with extragranular portion containing microcrystalline cellulose, copovidone, colloidal silicon dioxide, sodium stearyl fumarate for lubrication and compressed to get multiple unit pellet system. Extragranular materials may or may not be granulated before blending with the pellets.

The present invention is to provide extended release pellets which are prepared by coating active pharmaceutical ingredient spheres with seal coating of approximately 5% buildup using Opadry clear, hypromellose or povidone and purified water, functional coating of approximately 50% buildup using Surelease with or without hypromellose and purified water, seal coating of approximately 5% buildup over functional coated pellets using Opadry clear, hypromellose or povidone and purified water. Obtained pellets were blended with extragranular portion containing microcrystalline cellulose, copovidone, colloidal silicon dioxide, sodium stearyl fumarate for lubrication and compressed to get multiple unit pellet system. Extragranular materials may or may not be granulated before blending with the pellets.

The tablet obtained is optionally coated with Opadry ready to use film coating is selected from and not limited to Opadry II® (Y-30-13579-A), Opadry Clear® (YS-2-19114-A), Opadry II white, Opadry II brown, Opadry 11 orange, or Opadry II yellow.

The following examples describes the nature of the invention and are given only for the purpose of illustrating the present invention in more detail and are not limitative and relate to solutions, which have been particularly effective on bench scale.

### Example 1

| | | |
|---|---|---|
| | | Concentration |
| S. No | Ingredients | (mg/unit) |

Extrusion spheronization

| | | |
|---|---|---|
| 1. | Metformin hydrochloride | 500.00 |
| 2. | Microcrystalline cellulose (Avicel PH 101) | 50.00 |
| 3. | Povidone K-30 | 16.00 |

| | |
|---|---|
| Total weight of extruded spheres | 566.0 |

Seal coating

| | | |
|---|---|---|
| 4. | PEG 4000 | 28.00 |
| 5. | Purified water | Q.S. |

| | |
|---|---|
| Total weight of seal coated pellets | 594.00 |

Functional coating

| | | |
|---|---|---|
| 6. | Ethyl cellulose 10 cps | 213.0 |
| 7. | Hypromellose E5 LV | 24.0 |
| 8. | Triethyl citrate | 30.0 |
| 9. | Talc | 30.0 |
| 10. | Isopropyl alcohol | Q.S. |
| 11. | Purified water | Q.S. |

| | |
|---|---|
| Total weight of functional coated pellets | 891.00 |

Seal coating

| | | |
|---|---|---|
| 12. | PEG 4000 | 44.00 |
| 13. | Purified water | Q.S. |

| | |
|---|---|
| Total weight of final coated pellets | 935.00 |

Extragranular portion

| | | |
|---|---|---|
| 14. | Microcrystalline cellulose (Ceolus Kg 1000) | 50.00 |
| 15. | Copovidone (Kollidon VA 64) | 50.00 |
| 16. | Colloidal silicon dioxide (Aerosil 200 P) | 10.00 |
| 17. | Magnesium stearate | 10.00 |

| | |
|---|---|
| Total weight of tablet | 1055.00 |

### Manufacturing Process:

Metformin hydrochloride, Microcrystalline cellulose (Avicel PH 101) and Povidone K-30 were granulated, extruded and spheronized to form Metformin hydrochloride spheres. Seal coated pellets were prepared by coating Metformin hydrochloride spheres with seal coating of approx. 5% buildup using PEG 4000 and Purified water. Functional coating of approx. 50% buildup was prepared by mixing Ethyl cellulose 10 cps, Hypromellose E5 LV, Triethyl citrate, Talc, Isopropyl alcohol and Purified water and coated over seal coated Metformin hydrochloride spheres. Seal coating of approx. 5% buildup was prepared by PEG 4000 and Purified water and coated over functional coated Metformin hydrochloride spheres to form final coated pellets. Obtained pellets were blended with extragranular portion containing Microcrystalline cellulose (Ceolus Kg 1000), Copovidone (Kollidon VA 64), Colloidal silicon dioxide (Aerosil 200 P), Magnesium stearate for lubrication and compressed to get Metformin hydrochloride extended release multiple unit pellet system.

### Example 2

| | | |
|---|---|---|
| | | Concentration |
| S. No | Ingredients | (mg/unit) |

Extrusion spheronization

| | | |
|---|---|---|
| 1. | Metformin hydrochloride | 500.00 |
| 2. | Microcrystalline cellulose (Avicel PH 101) | 50.00 |
| 3. | Hydroxy propyl cellulose | 25.00 |

| | |
|---|---|
| Total weight of extruded spheres | 575.00 |

Seal coating

| | | |
|---|---|---|
| 4. | Hypromellose E5 LV | 58.00 |
| 5. | Purified water | Q.S. |

| | |
|---|---|
| Total weight of seal coated pellets | 633.00 |

Functional coating

| | | |
|---|---|---|
| 6. | Ethyl cellulose 10 cps | 138.00 |
| 7. | Dibutyl sebacate | 17.25 |
| 8. | Talc | 17.25 |
| 9. | Isopropyl alcohol | Q.S. |
| 10. | Purified water | Q.S. |

| | |
|---|---|
| Total weight of functional coated pellets | 806.0 |

Seal coating

| | | |
|---|---|---|
| 11. | Hypromellose E5 LV | 44.00 |
| 12. | Purified water | Q.S. |

| | |
|---|---|
| Total weight of final coated pellets | 850.00 |

Extragranular portion

| | | |
|---|---|---|
| 13. | Microcrystalline cellulose (Ceolus Kg 1000) | 50.00 |
| 14. | Copovidone (Kollidon VA 64) | 30.00 |
| 15. | Colloidal silicon dioxide (Aerosil 200 P) | 10.00 |
| 16. | Magnesium stearate | 10.00 |

| | |
|---|---|
| Total weight of tablet | 950.00 |

Metformin hydrochloride, Lactose and Hydroxy propyl cellulose were granulated, extruded and spheronized to form Metformin Hydrochloride spheres. Seal coated pellets were prepared by coating Metformin hydrochloride spheres with seal coating of approx. 10% buildup using Hypromellose E5 LV and Purified water. Functional coating of approx. 50% buildup was prepared by mixing Ethyl cellulose 10 cps, Dibutyl sebacate, Talc, Isopropyl alcohol and Purified water and coated over seal coated Metformin hydrochloride spheres. Seal coating of approx. 5% buildup was prepared by Hypromellose E5 LV and Purified water and coated over functional coated Metformin hydrochloride spheres to form final coated pellets. Obtained pellets were blended with extragranular portion containing Microcrystalline cellulose (Ceolus Kg 1000), Copovidone (Kollidon VA 64), Colloidal silicon dioxide (Aerosil 200 P), Magnesium stearate for lubrication and compressed to get Metformin hydrochloride extended release multiple unit pellet system.

### Example 3

| | | |
|---|---|---|
| | | Concentration |
| S. No | Ingredients | (mg/unit) |

Extrusion spheronization

| | | |
|---|---|---|
| 1. | Metformin hydrochloride | 500.00 |
| 2. | Microcrystalline cellulose (Ceolus KG 1000) | 10.00 |
| 3. | Copovidone (Kollidon VA 64) | 10.00 |

| | |
|---|---|
| Total weight of pellets | 520.00 |

Seal coating

| | | |
|---|---|---|
| 4. | Eudragit NM 30 D | 26.00 |
| 5. | Purified water | Q.S. |

| | |
|---|---|
| Total weight of seal coated pellets | 546.00 |

Functional coating

| | | |
|---|---|---|
| 6. | Ethyl cellulose 10 cps | 196.00 |
| 7. | Hypromellose E5 LV | 22.00 |
| 8. | Dibutyl sebacate | 28.00 |
| 9. | Talc | 28.00 |
| 10. | Isopropyl alcohol | Q.S. |
| 11. | Purified water | Q.S. |

| | |
|---|---|
| Total weight of functional coated pellets | 820.00 |

Seal coating

| | | |
|---|---|---|
| 12. | PEG 6000 | 44.00 |
| 13. | Purified water | Q.S. |

| | |
|---|---|
| Total weight of final coated pellets | 864.00 |

Extragranular portion

| | | |
|---|---|---|
| 14. | Microcrystalline cellulose (Ceolus Kg 711) | 30.00 |
| 15. | Copovidone (Kollidon VA 64) | 40.00 |
| 16. | Colloidal silicon dioxide (Aerosil 200 P) | 5.00 |
| 17. | Sodium stearyl fumarate | 10.00 |

| | |
|---|---|
| Total weight of tablet | 949.00 |

Metformin hydrochloride, Microcrystalline cellulose and Copovidone were extruded and spheronized to form Metformin hydrochloride pellets. Seal coated pellets were prepared by coating Metformin Hydrochloride spheres with seal coating of approx. 5% buildup using Eudragit NM 30 D and Purified water. Functional coating of approx. 50% buildup was prepared by mixing Ethyl cellulose 10 cps, Hypromellose E5 LV, Dibutyl sebacate, Talc, Isopropyl alcohol and Purified water and coated over seal coated Metformin hydrochloride spheres. Seal coating of approx. 5% buildup was prepared by PEG 6000 and Purified water and coated over functional coated Metformin hydrochloride spheres to form final coated pellets. Obtained pellets were blended with extragranular portion containing Microcrystalline cellulose (Ceolus Kg 711), Copovidone (Kollidon VA 64), Colloidal silicon dioxide (Aerosil 200 P), Sodium stearyl fumarate for lubrication and compressed to get Metformin hydrochloride extended release multiple unit pellet system.

### Example 4

| | | |
|---|---|---|
| | | Concentration |
| S. No | Ingredients | (mg/unit) |

Extrusion spheronization

| | | |
|---|---|---|
| 1. | Metformin Hydrochloride | 500.00 |
| 2. | Microcrystalline cellulose (Ceolus KG 1000) | 10.00 |
| 3. | Hydroxy propyl cellulose (Klucel LF) | 12.00 |

| | |
|---|---|
| Total weight of pellets | 522.00 |

Seal coating

| | | |
|---|---|---|
| 4. | Opadry clear (03K19229) | 15.80 |
| 5. | Purified water | Q.S. |

| | |
|---|---|
| Total weight of seal coated pellets | 537.80 |

Functional coating

| | | |
|---|---|---|
| 6. | Surelease E-7-19040 | 234.00 |
| 7. | Hypromellose E5 LV | 26.00 |
| 8. | Purified water | Q.S. |

| | |
|---|---|
| Total weight of functional coated pellets | 797.80 |

Seal coating

| | | |
|---|---|---|
| 9. | Opadry clear (03K19229) | 15.80 |
| 10. | Purified water | Q.S. |

| | |
|---|---|
| Total weight of final coated pellets | 813.60 |

Extragranular portion

| | | |
|---|---|---|
| 11. | Microcrystalline cellulose (Ceolus Kg 711) | 50.00 |
| 12. | Copovidone (Kollidon VA 64) | 50.00 |
| 13. | Colloidal silicon dioxide (Aerosil 200 P) | 8.40 |
| 14. | Sodium stearyl fumarate | 10.00 |

| | |
|---|---|
| Total weight of tablet | 932.00 |

### Manufacturing Process:

Metformin hydrochloride, Microcrystalline cellulose and hydroxyl propyl cellulose were extruded and spheronized to form Metformin hydrochloride pellets. Seal coated pellets were prepared by coating Metformin hydrochloride spheres with seal coating of approx. 5% buildup using Opadry clear and Purified water. Functional coating of approx. 50% buildup was prepared by mixing Surelease E-7-19040, Hypromellose E5 LV, and Purified water and coated over seal coated Metformin hydrochloride spheres. Seal coating of approx. 5% buildup was prepared by Opadry clear and Purified water and coated over functional coated Metformin hydrochloride spheres to form final coated pellets. Obtained pellets were blended with extragranular portion containing Microcrystalline cellulose (Ceolus Kg 711), Copovidone (Kollidon VA 64), Colloidal silicon dioxide (Aerosil 200 P), Sodium stearyl fumarate for lubrication and compressed to get Metformin hydrochloride extended release multiple unit pellet system.

### Example 5

| | | |
|---|---|---|
| | | Concentration |
| S. No | Ingredients | (mg/unit) |

Extrusion spheronization

| | | |
|---|---|---|
| 1. | Metformin hydrochloride | 500.00 |
| 2. | Microcrystalline cellulose (Ceolus KG 1000) | 10.00 |
| 3. | Hydroxy propyl cellulose (Klucel LF) | 10.00 |

| | |
|---|---|
| Total weight of pellets | 520.00 |

Seal coating

| | | |
|---|---|---|
| 4. | Opadry clear (03K19229) | 26.00 |
| 5. | Purified water | Q.S. |

| | |
|---|---|
| Total weight of seal coated pellets | 546.00 |

Functional coating

| | | |
|---|---|---|
| 6. | Ethyl cellulose 10 cps | 177.00 |
| 7. | Hypromellose E5 LV | 13.00 |
| 8. | Dibutyl sebacate | 32.00 |
| 9. | Talc | 32.00 |
| 10. | Isopropyl alcohol | Q.S. |
| 11. | Purified water | Q.S. |

| | |
|---|---|
| Total weight of functional coated pellets | 800.00 |

Seal coating

| | | |
|---|---|---|
| 12. | Opadry clear (03K19229) | 26.00 |
| 13. | Purified water | Q.S. |

| | |
|---|---|
| Total weight of final coated pellets | 826.00 |

Extragranular portion

| | | |
|---|---|---|
| 14. | Microcrystalline cellulose (Ceolus Kg 711) | 50.00 |
| 15. | Copovidone (Kollidon VA 64) | 45.60 |
| 16. | Colloidal silicon dioxide (Aerosil 200 P) | 5.00 |
| 17. | Sodium stearyl fumarate | 10.00 |

| | |
|---|---|
| Total weight of tablet | 937.0 |

### Manufacturing Process:

Metformin hydrochloride, Microcrystalline cellulose and Hydroxyl propyl cellulose were extruded and spheronized to form Metformin hydrochloride pellets. Seal coated pellets were prepared by coating Metformin hydrochloride spheres with seal coating of approx. 5% buildup using Opadry clear and Purified water. Functional coating of approx. 50% buildup was prepared by mixing Ethylcellulose 100 cps, Hypromellose E5 LV, Dibutyl sebacate, Talc, Isopropyl alcohol and Purified water and coated over seal coated Metformin hydrochloride spheres. Seal coating of approx. 5% buildup was prepared by Opadry clear and Purified water and coated over functional coated Metformin hydrochloride spheres to form final coated pellets. Obtained pellets were blended with extragranular portion containing Microcrystalline cellulose (Ceolus Kg 711), Copovidone (Kollidon VA 64), Colloidal silicon dioxide (Aerosil 200 P), Sodium stearyl fumarate for lubrication and compressed to get Metformin hydrochloride extended release multiple unit pellet system.

### Example 6

| | | |
|---|---|---|
| | | Concentration |
| S. No | Ingredients | (mg/unit) |

Extrusion spheronization

| | | |
|---|---|---|
| 1. | Venlafaxine hydrochloride | 150.00 |
| 2. | Microcrystalline cellulose (Avicel PH 101) | 50.00 |
| 3. | Povidone K-30 | 16.00 |

| | |
|---|---|
| Total weight of extruded spheres | 216.00 |

Seal coating

| | | |
|---|---|---|
| 4. | PEG 4000 | 10.00 |
| 5. | Purified water | Q.S. |

| | |
|---|---|
| Total weight of seal coated pellets | 226.00 |

Functional coating

| | | |
|---|---|---|
| 6. | Ethyl cellulose 10 cps | 81.40 |
| 7. | Hypromellose E5 LV | 9.04 |
| 8. | Triethyl citrate | 4.52 |
| 9. | Talc | 4.52 |
| 10. | Isopropyl alcohol | Q.S. |
| 11. | Purified water | Q.S. |

| | |
|---|---|
| Total weight of functional coated pellets | 325.48 |

Seal coating

| | | |
|---|---|---|
| 12. | PEG 4000 | 10.00 |
| 13. | Purified water | Q.S. |

| | |
|---|---|
| Total weight of final coated pellets | 335.48 |

Extragranular portion

| | | |
|---|---|---|
| 14. | Microcrystalline cellulose (Ceolus Kg 1000) | 50.00 |
| 15. | Copovidone (Kollidon VA 64) | 30.00 |
| 16. | Colloidal silicon dioxide (Aerosil 200 P) | 10.00 |
| 17. | Magnesium stearate | 9.42 |

| | |
|---|---|
| Total weight of tablet | 435.0 |

Venlafaxine hydrochloride, Microcrystalline cellulose (Avicel PH 101) and Povidone K-30 were granulated, extruded and spheronized to form Venlafaxine hydrochloride spheres. Seal coated pellets were prepared by coating Venlafaxine hydrochloride spheres with seal coating of approx. 5% buildup using PEG 4000 and Purified water. Functional coating of approx. 50% buildup was prepared by mixing Ethyl cellulose 10 cps, Hypromellose E5 LV, Triethyl citrate, Talc, Isopropyl alcohol and Purified water and coated over seal coated Venlafaxine hydrochloride spheres. Seal coating of approx. 5% buildup was prepared by PEG 4000 and Purified water and coated over Functional coated Venlafaxine hydrochloride spheres to form final coated pellets. Obtained pellets were blended with Extragranular portion containing Microcrystalline cellulose (Ceolus Kg 1000), Copovidone (Kollidon VA 64), Colloidal silicon dioxide (Aerosil 200 P), Magnesium stearate for lubrication and compressed to get Venlafaxine hydrochloride extended release multiple unit pellet system.

### Example 7

| | | |
|---|---|---|
| | | Concentration |
| S. No | Ingredients | (mg/unit) |

Extrusion spheronization

| | | |
|---|---|---|
| 1. | Metoprolol succinate | 190.00 |
| 2. | Microcrystalline cellulose (Avicel PH 101) | 100.00 |
| 3. | Hydroxy Propyl Cellulose (Klucel LF) | 5.00 |

| | |
|---|---|
| Total weight of extruded spheres | 295.00 |

Seal coating

| | | |
|---|---|---|
| 4. | PEG 4000 | 15.00 |
| 5. | Purified water | Q.S. |

| | |
|---|---|
| Total weight of seal coated pellets | 310.00 |

Functional coating

| | | |
|---|---|---|
| 6. | Ethyl cellulose 10 cps | 74.00 |
| 7. | Hypromellose E5 LV | 50.00 |
| 8. | Triethyl citrate | 15.00 |
| 9. | Talc | 15.00 |
| 10. | Isopropyl alcohol | Q.S. |
| 11. | Purified water | Q.S. |

| | |
|---|---|
| Total weight of functional coated pellets | 464.00 |

Seal coating

| | | |
|---|---|---|
| 12. | PEG 4000 | 24.00 |
| 13. | Purified water | Q.S. |

| | |
|---|---|
| Total weight of final coated pellets | 488.00 |

Extragranular portion

| | | |
|---|---|---|
| 14. | Microcrystalline cellulose (Ceolus Kg 1000) | 232.00 |
| 15. | Copovidone (Kollidon VA 64) | 60.00 |
| 16. | Colloidal silicon dioxide (Aerosil 200 P) | 10.00 |
| 17. | Magnesium stearate | 10.00 |

| | |
|---|---|
| Total weight of tablet | 800.00 |

Optional Film coating

| | |
|---|---|
| Opadry ready to use film coating | 24.00 |

### Manufacturing Process:

Metoprolol succinate, Microcrystalline cellulose (Avicel PH 101) and Hydroxy Propyl Cellulose (Klucel LF) were granulated, extruded and spheronized to form Metoprolol succinate spheres. Seal coated pellets were prepared by coating Metoprolol succinate spheres with seal coating of approx. 5% buildup using PEG 4000 and Purified water. Functional coating of approx. 50% buildup was prepared by mixing Ethyl cellulose 10 cps, Hypromellose E5 LV, Triethyl citrate, Talc, Isopropyl alcohol and Purified water and coated over seal coated Metoprolol succinate spheres. Seal coating of approx. 5% buildup was prepared by PEG 4000 and Purified water and coated over Functional coated Metoprolol succinate spheres to form final coated pellets. Obtained pellets were blended with Extragranular portion containing Microcrystalline cellulose (Ceolus Kg 1000), Copovidone (Kollidon VA 64), Colloidal silicon dioxide (Aerosil 200 P), Magnesium stearate for lubrication and compressed to get Metoprolol extended release multiple unit pellet system. The compressed MUPS are optionally coated using film coating solution of Opadry.

### Example 8

| | | |
|---|---|---|
| | | Concentration |
| S. No | Ingredients | (mg/unit) |

Extrusion spheronization

| | | |
|---|---|---|
| 1. | Metoprolol succinate | 190.00 |
| 2. | Microcrystalline cellulose (Avicel PH 101) | 97.00 |
| 3. | Povidone K-30 | 8.00 |

| | |
|---|---|
| Total weight of extruded spheres | 295.00 |

Seal coating

| | | |
|---|---|---|
| 4. | PEG 4000 | 15.00 |
| 5. | Purified water | Q.S. |

| | |
|---|---|
| Total weight of seal coated pellets | 310.00 |

Functional coating

| | | |
|---|---|---|
| 6. | Ethyl cellulose 10 cps | 74.00 |
| 7. | Hypromellose E5 LV | 50.00 |
| 8. | Triethyl citrate | 15.00 |
| 9. | Talc | 15.00 |
| 10. | Isopropyl alcohol | Q.S. |
| 11. | Purified water | Q.S. |

| | |
|---|---|
| Total weight of functional coated pellets | 464.00 |

Seal coating

| | | |
|---|---|---|
| 12. | PEG 4000 | 24.00 |
| 13. | Purified water | Q.S. |

| | |
|---|---|
| Total weight of final coated pellets | 488.00 |

Extragranular portion

| | | |
|---|---|---|
| 14. | Microcrystalline cellulose (Ceolus Kg 1000) | 232.00 |
| 15. | Copovidone (Kollidon VA 64) | 60.00 |
| 16. | Colloidal silicon dioxide (Aerosil 200 P) | 10.00 |
| 17 | Magnesium stearate | 10.00 |

| | |
|---|---|
| Total weight of tablet | 800.00 |

### Manufacturing Process:

Metoprolol succinate, Microcrystalline cellulose (Avicel PH 101) and Povidone were granulated, extruded and spheronized to form Metoprolol succinate spheres. Seal coated pellets were prepared by coating Metoprolol succinate spheres with seal coating of approx. 5% buildup using PEG 4000 and Purified water. Functional coating of approx. 50% buildup was prepared by mixing Ethyl cellulose 10 cps, Hypromellose E5 LV, Triethyl citrate, Talc, Isopropyl alcohol and Purified water and coated over seal coated Metoprolol succinate spheres. Seal coating of approx. 5% buildup was prepared by PEG 4000 and Purified water and coated over Functional coated Metoprolol succinate spheres to form final coated pellets. Obtained pellets were blended with extragranular portion containing Microcrystalline cellulose (Ceolus Kg 1000), Copovidone (Kollidon VA 64), Colloidal silicon dioxide (Aerosil 200 P), Magnesium stearate for lubrication and compressed to get Metoprolol succinate extended release multiple unit pellet system.

### Example 9

| | | |
|---|---|---|
| | | Concentration |
| S. No | Ingredients | (mg/unit) |

Extrusion spheronization

| | | |
|---|---|---|
| 1. | Metoprolol Succinate | 190.00 |
| 2. | Microcrystalline cellulose (Avicel PH 101) | 100.00 |
| 3. | Hydroxy Propyl Cellulose (Klucel LF) | 5.00 |

| | |
|---|---|
| Total weight of extruded spheres | 295.00 |

Seal coating

| | | |
|---|---|---|
| 4. | PEG 4000 | 15.00 |
| 5. | Purified water | Q.S. |

| | |
|---|---|
| Total weight of seal coated pellets | 310.00 |

Functional coating

| | | |
|---|---|---|
| 6. | Ethyl cellulose 10 cps | 111.00 |
| 7. | Hypromellose E5 LV | 13.00 |
| 8. | Triethyl citrate | 16.00 |
| 9. | Talc | 16.00 |
| 10. | Isopropyl alcohol | Q.S. |
| 11. | Purified water | Q.S. |

| | |
|---|---|
| Total weight of functional coated pellets | 466.00 |

Seal coating

| | | |
|---|---|---|
| 12. | PEG 4000 | 24.00 |
| 13. | Purified water | Q.S. |

| | |
|---|---|
| Total weight of final coated pellets | 490.00 |

Extragranular portion

| | | |
|---|---|---|
| 14. | Microcrystalline cellulose (Ceolus Kg 1000) | 150.00 |
| 15. | Copovidone (Kollidon VA 64) | 50.00 |
| 16. | Colloidal silicon dioxide (Aerosil 200 P) | 10.00 |
| 17. | Magnesium stearate | 10.00 |

| | |
|---|---|
| Total weight of tablet | 710.00 |

### Manufacturing Process:

Metoprolol succinate, Microcrystalline cellulose (Avicel PH 101) and Hydroxy Propyl Cellulose (Klucel LF) were granulated, extruded and spheronized to form Metoprolol succinate spheres. Seal coated pellets were prepared by coating Metoprolol succinate spheres with seal coating of approx. 5% buildup using PEG 4000 and Purified water. Functional coating of approx. 50% buildup was prepared by mixing Ethyl cellulose 10 cps, Hypromellose E5 LV, Triethyl citrate, Talc, Isopropyl alcohol and Purified water and coated over seal coated Metoprolol succinate spheres. Seal coating of approx. 5% buildup was prepared by PEG 4000 and Purified water and coated over functional coated Metoprolol succinate spheres to form final coated pellets. Obtained pellets were blended with Extragranular portion containing Microcrystalline cellulose (Ceolus Kg 1000), Copovidone (Kollidon VA 64), Colloidal silicon dioxide (Aerosil 200 P), Magnesium stearate for lubrication and compressed to get Metoprolol extended release multiple unit pellet system.

### Example 10

| | | |
|---|---|---|
| | | Concentration |
| S. No | Ingredients | (mg/unit) |

Extrusion spheronization

| | | |
|---|---|---|
| 1. | Metoprolol succinate | 190.00 |
| 2. | Microcrystalline cellulose (Avicel PH 101) | 121.00 |
| 3. | Hydroxy Propyl Cellulose (Klucel LF) | 5.00 |

| | |
|---|---|
| Total weight of extruded spheres | 316.00 |

Seal coating

| | | |
|---|---|---|
| 4. | Opadry clear (03K19229) | 15.80 |
| 5. | Purified water | Q.S. |

| | |
|---|---|
| Total weight of seal coated pellets | 331.80 |

Functional coating

| | | |
|---|---|---|
| 6. | Surelease E-7-19040 | 142.20 |
| 7. | Hypromellose E5 LV | 15.80 |
| 8. | Purified water | Q.S. |

| | |
|---|---|
| Total weight of functional coated pellets | 489.80 |

Seal coating

| | | |
|---|---|---|
| 9. | Opadry clear (03K19229) | 15.80 |
| 10. | Purified water | Q.S. |

| | |
|---|---|
| Total weight of final coated pellets | 505.60 |

Extragranular portion (Granulation by FPB)

| | | |
|---|---|---|
| 11. | Microcrystalline cellulose (Ceolus Kg 802) | 150.00 |
| 12. | Copovidone (Kollidon VA 64) | 50.00 |
| 13. | Purified water | Q.S |

Pre-lubrication and lubrication

| 14. | Colloidal silicon dioxide (Aerosil 200 P) | 10.00 |
|---|---|---|
| 15. | Magnesium stearate | 9.00 |
| Core weight of tablet | | 725.00 |
| 16. | Film coating | 17.40 |

| | |
|---|---|
| Film coated weight of tablet | 742.00 |

### Manufacturing Process:

Metoprolol succinate, Microcrystalline cellulose (Avicel PH 101) and Hydroxy Propyl Cellulose (Klucel LF) were granulated, extruded and spheronized to form Metoprolol succinate spheres. Seal coated pellets were prepared by coating Metoprolol succinate spheres with seal coating of approx. 5% buildup using Opadry clear and Purified water. Functional coating of approx. 50% buildup was prepared by mixing Surelease E-7-19040, Hypromellose E5 LV and Purified water and coated over seal coated Metoprolol succinate spheres. Seal coating of approx. 5% buildup was prepared by Opadry clear and Purified water and coated over functional coated Metoprolol succinate spheres to form final coated pellets. Obtained pellets were granulated with extragranular material Microcrystalline cellulose (Ceolus Kg 802), Copovidone (Kollidon VA 64) by FBP process with top spray granulation to obtain uniform distribution of pellets and blended. Pre-lubrication done with Colloidal silicon dioxide (Aerosil 200 P), Magnesium stearate for lubrication and compressed to get Metoprolol extended release multiple unit pellet system. Further tablets film coated with film coating material

### Example 11

| | | |
|---|---|---|
| | | Concentration |
| S. No | Ingredients | (mg/unit) |

Extrusion spheronization

| | | |
|---|---|---|
| 1. | Metoprolol succinate | 190.00 |
| 2. | Microcrystalline cellulose (Avicel PH 101) | 121.00 |
| 3. | Hydroxy Propyl Cellulose (Klucel LF) | 5.00 |

| | |
|---|---|
| Total weight of extruded spheres | 316.00 |

Seal coating

| | | |
|---|---|---|
| 4. | Opadry clear (03K19229) | 15.80 |
| 5. | Purified water | Q.S. |

| | |
|---|---|
| Total weight of seal coated pellets | 331.80 |

Functional coating

| | | |
|---|---|---|
| 6. | Surelease E-7-19040 | 158.00 |
| 7. | Purified water | Q.S. |

| | |
|---|---|
| Total weight of functional coated pellets | 489.80 |

Seal coating

| | | |
|---|---|---|
| 8. | Opadry clear (03K19229) | 15.80 |
| 9. | Purified water | Q.S. |

| | |
|---|---|
| Total weight of final coated pellets | 505.60 |

Extragranular portion

| | | |
|---|---|---|
| 10. | Microcrystalline cellulose (Ceolus Kg 1000) | 150.00 |
| 11. | Copovidone (Kollidon VA 64) | 50.00 |
| 12. | Colloidal silicon dioxide (Aerosil 200 P) | 10.00 |
| 13. | Magnesium stearate | 9.40 |

| | |
|---|---|
| Total weight of tablet | 725.00 |

### Manufacturing Process:

Metoprolol succinate, Microcrystalline cellulose (Avicel PH 101) and Hydroxy Propyl Cellulose (Klucel LF) were granulated, extruded and spheronized to form Metoprolol succinate spheres. Seal coated pellets were prepared by coating Metoprolol succinate spheres with seal coating of approx. 5% buildup using Opadry clear and Purified water. Functional coating of approx. 50% buildup was prepared by mixing Surelease E-7-19040 and Purified water and coated over seal coated Metoprolol succinate spheres. Seal coating of approx. 5% buildup was prepared by Opadry clear and Purified water and coated over Functional coated Metoprolol succinate spheres to form final coated pellets. Extragranular portion containing Microcrystalline cellulose (Ceolus Kg 1000), Copovidone (Kollidon VA 64), Colloidal silicon dioxide (Aerosil 200 P) were granulated using FBP process. Obtained pellets were blended with granulated Extragranular portion and Magnesium stearate for lubrication and compressed to get Metoprolol extended release multiple unit pellet system.

### Example 12

| | | |
|---|---|---|
| | | Concentration |
| S. No | Ingredients | (mg/unit) |

Extrusion spheronization

| | | |
|---|---|---|
| 1. | Metoprolol succinate | 190.00 |
| 2. | Microcrystalline cellulose (Avicel PH 101) | 121.00 |
| 3. | Hydroxy Propyl Cellulose (Klucel LF) | 5.00 |

| | |
|---|---|
| Total weight of extruded spheres | 316.00 |

Seal coating

| | | |
|---|---|---|
| 4. | Hypromellose E5 LV | 15.80 |
| 5. | Purified water | Q.S. |

| | |
|---|---|
| Total weight of seal coated pellets | 331.80 |

Functional coating

| | | |
|---|---|---|
| 6. | Ethyl cellulose 100 cps | 79.00 |
| 7. | Isopropyl alcohol | Q.S. |
| 8. | Purified water | Q.S. |

| | |
|---|---|
| Total weight of functional coated pellets | 410.80 |

Seal coating

| | | |
|---|---|---|
| 9. | Hypromellose E5 LV | 15.80 |
| 10. | Purified water | Q.S. |

| | |
|---|---|
| Total weight of final coated pellets | 426.60 |

Extragranular portion

| | | |
|---|---|---|
| 11. | Microcrystalline cellulose (Ceolus Kg 1000) | 150.00 |
| 12. | Copovidone (Kollidon VA 64) | 50.00 |
| 13. | Colloidal silicon dioxide (Aerosil 200 P) | 10.00 |
| 14. | Magnesium stearate | 9.40 |

| | |
|---|---|
| Total weight of tablet | 646.00 |

### Manufacturing Process:

Metoprolol succinate, Microcrystalline cellulose (Avicel PH 101) and Hydroxy Propyl Cellulose (Klucel LF) were granulated, extruded and spheronized to form Metoprolol succinate spheres. Seal coated pellets were prepared by coating Metoprolol succinate spheres with seal coating of approx. 5% buildup using Hypromellose E5 LV and Purified water. Functional coating of approx. 50% buildup was prepared by mixing ethylcellulose 100 cps, isopropyl alcohol and Purified water and coated over seal coated Metoprolol Succinate spheres. Seal coating of approx. 5% buildup was prepared by Hypromellose E5 LV and Purified water and coated over functional coated Metoprolol succinate spheres to form final coated pellets. Obtained pellets were blended with Extragranular portion containing Microcrystalline cellulose (Ceolus Kg 1000), Copovidone (Kollidon VA 64), Colloidal silicon dioxide (Aerosil 200 P), Magnesium stearate for lubrication and compressed to get Metoprolol extended release multiple unit pellet system.

### Example 13

| | | Concentration |
|---|---|---|
| S. No | Ingredients | (mg/unit) |
| 1. | Metformin hydrochloride | 500.00 |

| | |
|---|---|
| Total weight of pellets | 500.00 |

Seal coating

| | | |
|---|---|---|
| 2. | Opadry clear (03K19229) | 26.00 |
| 3. | Purified water | Q.S. |

| | |
|---|---|
| Total weight of seal coated pellets | 526.00 |

Functional coating

| | | |
|---|---|---|
| 4. | Ethyl cellulose 10 cps | 167.00 |
| 5. | Hypromellose E5 LV | 14.00 |
| 6. | Dibutyl sebacate | 28.00 |
| 7. | Talc | 28.00 |
| 8. | Isopropyl alcohol | Q.S. |
| 9. | Purified water | Q.S. |

| | |
|---|---|
| Total weight of functional coated pellets | 763.00 |

Seal coating

| | | |
|---|---|---|
| 10. | Opadry clear (03K19229) | 26.00 |
| 11. | Purified water | Q.S. |

| | |
|---|---|
| Total weight of final coated pellets | 789.00 |

Extragranular portion

| | | |
|---|---|---|
| 12. | Microcrystalline cellulose (Ceolus Kg 711) | 50.00 |
| 13. | Copovidone (Kollidon VA 64) | 45.60 |
| 14. | Colloidal silicon dioxide (Aerosil 200 P) | 5.00 |
| 15. | Sodium stearyl fumarate | 10.00 |

| | |
|---|---|
| Total weight of tablet | 899.6 |

### Manufacturing Process:

Metformin hydrochloride was spheronized to form Metformin hydrochloride pellets. Seal coated pellets were prepared by coating Metformin hydrochloride spheres with seal coating of approx. 5% buildup using Opadry clear and Purified water. Functional coating of approx. 50% buildup was prepared by mixing Ethylcellulose 100 cps, Hypromellose E5 LV, Dibutyl sebacate, Talc, Isopropyl alcohol and Purified water and coated over seal coated Metformin hydrochloride spheres. Seal coating of approx. 5% buildup was prepared by Opadry clear and purified water and coated over functional coated Metformin hydrochloride spheres to form final coated pellets. Obtained pellets were blended with Extragranular portion containing Microcrystalline cellulose (Ceolus Kg 711), Copovidone (Kollidon VA 64), Colloidal silicon dioxide (Aerosil 200 P), Sodium stearyl fumarate for lubrication and compressed to get Metformin hydrochloride extended release multiple unit pellet system.

### Example 14

| | | |
|---|---|---|
| | | Concentration |
| S. No | Ingredients | (mg/unit) |

Extrusion spheronization

| | | |
|---|---|---|
| 1. | Metoprolol succinate | 190.00 |
| 2. | Microcrystalline cellulose (Avicel PH 101) | 121.00 |
| 3. | Hydroxy Propyl Cellulose (Klucel LF) | 5.00 |

| | |
|---|---|
| Total weight of extruded spheres | 316.00 |

Seal coating

| | | |
|---|---|---|
| 4. | Povidone | 10.00 |
| 5. | Purified water | Q.S. |

| | |
|---|---|
| Total weight of seal coated pellets | 326.0 |

Functional coating

| | | |
|---|---|---|
| 6. | Ethyl cellulose 100 cps | 79.00 |
| 7. | Isopropyl alcohol | Q.S. |
| 8. | Purified water | Q.S. |

| | |
|---|---|
| Total weight of functional coated pellets | 405.0 |

Seal coating

| | | |
|---|---|---|
| 9. | Hypromellose E5 LV | 10.00 |
| 10. | Purified water | Q.S. |

| | |
|---|---|
| Total weight of final coated pellets | 415.0 |

Extragranular portion

| | | |
|---|---|---|
| 11. | Microcrystalline cellulose (Ceolus Kg 1000) | 150.00 |
| 12. | Copovidone (Kollidon VA 64) | 50.00 |
| 13. | Colloidal silicon dioxide (Aerosil 200 P) | 10.00 |
| 14. | Magnesium stearate | 9.40 |

| | |
|---|---|
| Total weight of tablet | 634.4 |

### Manufacturing Process:

Metoprolol succinate, Microcrystalline cellulose (Avicel PH 101) and Hydroxy Propyl Cellulose (Klucel LF) were granulated, extruded and spheronized to form Metoprolol succinate spheres. Seal coated pellets were prepared by coating Metoprolol succinate spheres with seal coating of approx. 5% buildup using Povidone and Purified water. Functional coating of approx. 50% buildup was prepared by mixing ethylcellulose 100 cps, isopropyl alcohol and Purified water and coated over seal coated Metoprolol Succinate spheres. Seal coating of approx. 5% buildup was prepared by Hypromellose E5 LV and Purified water and coated over Functional coated Metoprolol succinate spheres to form final coated pellets. Obtained pellets were blended with extragranular portion containing Microcrystalline cellulose (Ceolus Kg 1000), Copovidone (Kollidon VA 64), Colloidal silicon dioxide (Aerosil 200 P), Magnesium stearate for lubrication and compressed to get Metoprolol extended release multiple unit pellet system.

### Dissolution Data

TABLE 1
Dissolution data of Metformin hydrochloride
ER Tablets as given in Example 4:
pH 6.8 Phosphate buffer, Paddle, 900 ml

| Time points (hr) | Test product (% release) |
|---|---|
| 0.5 | 19 |
| 1 | 25 |
| 2 | 56 |
| 3 | 72 |
| 4 | 85 |
| 6 | 90 |
| 8 | 95 |
| 12 | 98 |

TABLE 2
Dissolution data of Metoprolol succinate
ER Tablets as given in Example 9
pH 6.8 Phosphate buffer, Paddle, 900 ml

| Time points (hr) | Test product (% release) |
|---|---|
| 1 | 15 |
| 2 | 20 |
| 4 | 36 |
| 8 | 58 |
| 10 | 65 |
| 12 | 79 |
| 20 | 95 |

TABLE 3
Dissolution data of Metoprolol succinate
ER Tablets as given in Example 11:
pH 6.8 Phosphate buffer, Paddle, 900 ml

| Time points (hr) | Test product (% release) |
|---|---|
| 1 | 10 |
| 2 | 19 |
| 4 | 31 |
| 8 | 52 |
| 10 | 60 |
| 12 | 71 |
| 20 | 90 |

### Stability Data

TABLE 4
stability data of Metformin hydrochloride ER Tablets of Example 4

| Sr. No. | Tests | | | Initial | 6 month |
|---|---|---|---|---|---|
| 1 | Appearance | | | White to off | White to off |
| | | | | white tablets | white tablets |
| 2 | Dissolution (%) | 0.5 | hr | 19 | 23 |
| | | 3 | hr | 72 | 76 |
| | | 8 | hr | 95 | 97 |

| 3 | Water content (%) | 4.5 | 5.1 |
|---|---|---|---|
| 4 | Related Substances | | |
| | Any unspecified impurity (%) | 0.02 | 0.05 |
| | Total impurities (%) | 0.80 | 1.01 |
| 5 | Assay (%) | 98.0 | 99.1 |

TABLE 5
stability data of Metoprolol Succinate ER Tablets of Example 9

| Sr. No. | Tests | | | Initial | 6 month |
|---|---|---|---|---|---|
| 1 | Appearance | | | White to off | White to off |
| | | | | white tablets | white tablets |
| 2 | Dissolution (%) | 1 | hr | 15 | 16 |
| | | 8 | hr | 58 | 56 |
| | | 12 | hr | 79 | 82 |

| 3 | Water content (%) | 5.0 | 4.7 |
|---|---|---|---|
| 4 | Related Substances | | |
| | Any unspecified | 0.05 | 0.10 |
| | degradation product | | |
| | Total impurities | 0.08 | 0.13 |
| 5 | Assay (%) | 98.5 | 99.0 |

### Nitrosamine Testing Data

TABLE 6
Nitrosamine testing data

| Formulation | NDMA | |
|---|---|---|
| Conventional manufacturing process | 18 | ppb |
| Example 2 | 4 | ppb |
| Example 5 | 5 | ppb |

1. A composition of extended release multiple unit pellet system comprising:
(a) core spheres containing active pharmaceutical ingredient and optionally excipients,
(b) seal coating over the core containing coating polymer and one or more solvents,
(c) functional coating over seal coated pellets containing release controlling polymers and other excipients,
(d) second seal coating over the functional coated pellets containing coating polymer and one or more solvents, and
(e) extra-granular portion containing tabletting excipients at least one or more selected from categories of diluent, disintegrant, binder, glidant and lubricant.

2. The composition as claimed in claim 1, wherein excipients are release controlling polymers, diluents, coating polymers, disintegrants, plasticizers, binders, glidants, anti-tacking agents or anti-adherents and lubricants.

3. The composition as claimed in claim 1, wherein the active pharmaceutical ingredient is selected from anti-diabetic agents, antibacterial, antacids, analgesic and anti-inflammatory agents, anti-arrhythmic agents, antiprotozoal agents, anti-coagulants, antidepressants, anti-epileptic agents, antifungal agents, antihistamines, anti-hypertensive agents, anti-muscarinic agents, antineoplastic agents, antimetabolites, anti-migraine agents, anti-parkinson agents, antipsychotic, hypnotic and sedating agents, anti-stroke agents, antitussive, antivirals, cardiac inotropic agents, corticosteroids, diuretics, enzymes, gastro-intestinal agents, haemostatics, lipid regulating agents, local anaesthetics, opioid analgesics, parasympathomimetics, anti-dementia drugs, peptides, proteins, sex hormones, stimulating agents, vasodilators and combinations thereof.

4. The composition as claimed in claim 3, wherein the active pharmaceutical ingredient is a high soluble high dose active pharmaceutical ingredient selected from the group consisting of Metformin hydrochloride, Acebutolol hydrochloride, Amantadine hydrochloride, Aminocaproic acid, Aminophylline, Amodiaquine hydrochloride, Ascorbic acid, Carbenoxolone sodium, Cefuroxime sodium, Chloroquine phosphate, Chloroquine sulphate, Chlorpromazine hydrochloride, Ciprofloxacin hydrochloride, Cloxacillin sodium, Cycloserine, Diltiazem hydrochloride, Diethyl Carbamazine citrate, Doxycycline hydrochloride, Ethosuximide, Ferrous gluconate, Isoniazid, Levamisole hydrochloride, Lincomycin hydrochloride, Mebeverine hydrochloride, Mepyramine maleate, Metoprolol tartrate, Metoprolol succinate, Nicotinamide, Nicotinic acid, Oxprenolol hydrochloride, Oxytetracycline hydrochloride, Penicillamine, Pentobarbitone sodium, Phenoxy Methyl Penicillin K, Phenyloin sodium, Piperazine adipate, Procainamide hydrochloride, Pseudoephedrine hydrochloride, Quinalbarbitone sodium, Quinine bisulphate, Ranitidine hydrochloride, Sodium Amino salicylate, Sodium fusidate, Sodium valproate, Streptomycin sulphate, Tetracycline hydrochloride, Troxidone, Potassium chloride, Venlafaxine hydrochloride, Verapamil hydrochloride and their pharmaceutically acceptable salts, ester and hydrates.

5. The composition as claimed in claim 3, wherein the active pharmaceutical ingredient is a low soluble high dose active pharmaceutical ingredient or derivative thereof selected from the group consisting of Acetazolamide, Allopurinol, Atenolol, Carbamazepine, Cefadroxil, Cephalexin, Chloramphenicol, Cefuroxime axetil, Chlorthalidone, Cimetidine, Clarithromycin, Clofazemine, Curcuminoids And Non Curcuminoids, Dapsone, Diclofenac sodium, Diiodohydroxy quinolone, Diloxanide furoate, Disulfiram, Erythromycin, Erythromycin estolate, Erythromycin stearate, Thacrynic acid, Ethionamide, Ethopropazine hydrochloride, Ferrous fumarate, Fluconazole, Flurbiprofen, Furazolidone, Griseofulvin, Hydrochlorthiazide, Ibuprofen, Ketoconazole, Ketoprofen, Labetalol hydrochloride, Levodopa, Linezolid, Lithium carbonate, Magaldrate, Mebendazole, Mefenamic acid, Megestrol acetate, Mercaptopurine, Nalidixic acid, Niclosamide, Nitrofurantoin, Norfloxacin, Oxyphenbutazone, Paracetamol, Phenindione, Phenobarbitone, Phenylbutazone, Phenylsulphathiazole, Piperazine phosphate, Proguanil hydrochloride, Promethazine theoclate, Propylthiouracil, Posaconazole, Quinidine Sulphate, Quinine Sulphate, Quinidochlor, Rifampicin, Spironolactone, Succinylsulphathiazole, Sulphadiazine, Sulphadimethoxine, Sulphadimidine, Sulphafurazole, Sulphaphenazole, Thiabendazole, Tinidazole, Tolbutamide, Triamterene, Sulphamethoxazole and the like and their pharmaceutically acceptable salts, ester and hydrates.

6. The composition as claimed in claim 1, wherein the diluent is mannitol powder, spray dried mannitol, microcrystalline cellulose, lactose, dicalcium phosphate, tricalcium phosphate, starch, pregelatinized starch, compressible sugars, silicified microcrystalline cellulose, silicon dioxide, and calcium carbonate.

7. The composition as claimed in claim 1, wherein the binder is methylcellulose, hydroxypropyl cellulose, hydroxypropyl methylcellulose, povidone, copovidone, gelatin, gum arabic, ethyl cellulose, polyvinyl alcohol, starch, pregelatinized starch, agar, tragacanth or sodium alginate.

8. The composition as claimed in claim 1, wherein the coating polymer is sugars, zein, celluloses, hydroxypropyl celluloses, hydroxypropyl methylcelluloses, hydroxyethyl celluloses, polyvinyl alcohols, polyethylene glycols, poloxamers, methacryclic acid polymers, poly vinyl acetate, ethylcelluloses, gelatins, polyarginines, polyglycines, polyvinylpyrrolidones, vinyl acetate copolymers, Opdary coating mixtures and any mixtures thereof.

9. The composition as claimed in claim 1, wherein the release controlling polymer is ethyl cellulose, cellulose acetate, cellulose acetate butyrate, ethylene vinylacetate copolymer, polyvidone acetate, polyvinyl acetate, ethyl acrylate/methyl methacrylate copolymer, ammonia methacrylate copolymer, methylcellulose, hydroxy propyl cellulose, hypromellose or combinations thereof.

10. The composition as claimed in claim 2, wherein the plasticizer is polyethylene glycol, triethyl citrate, tributyl citrate, glycerin, dibutyl sebacate, glyceryl tricaprylate/caprate, medium chain triglyceride, oleic acid, triacetin and diethylphthalate.

11. The composition as claimed in claim 2, wherein the anti-adherent or anti-tacking agent is magnesium stearate, talc, calcium stearate, glyceryl behenate, polyethylene glycols, hydrogenated vegetable oil, mineral oil, stearic acid.

12. The composition as claimed in claim 1, wherein the disintegrant is croscarmellose sodium, microcrystalline cellulose, crospovidone, polacrilin potassium and sodium starch glycolate.

13. The composition as claimed in claim 1, wherein the glidant is colloidal silicon dioxide, magnesium trisilicate, powdered cellulose, talc, tribasic calcium phosphate and combinations thereof.

14. The composition as claimed in claim 1, wherein the lubricant is magnesium stearate, stearic acid, sodium stearyl fumarate, calcium stearate, hydrogenated vegetable oil, mineral oil, polyethylene glycol, polyethylene glycol 4000-6000, talc, and glyceryl behenate.

15. The composition as claimed in claim 1, wherein the solvent is isopropyl alcohol, acetone, methanol, ethanol, water and mixtures thereof.

16. (canceled)

17. (canceled)

18. (canceled)

19. The process for the preparation of extended release multiple unit pellet system composition as claimed in claim 1, comprising steps of:
(a) extruding and spheronizing active ingredient with excipients to form extruded spheres,
(b) mixing seal coating polymer with water or any other solvent and coating over extruded spheres obtained in step (a) to form seal coated pellets,
(c) adding and mixing release controlling polymers, anti-adherent, plasticizer in water or any other solvent under vigorous stirring and coating over seal coated pellets obtained in step (b) to form functional coated pellets,
(d) mixing seal coating polymer with water or any other solvent and coating over pellets obtained in step (c) to form final coated pellets,
(e) blending the obtained final coated pellets obtained in step (d) with diluent, disintegrant, binder, glidant, lubricant, and
(f) compressing the blended pellets obtained in step (e) into tablet or filling the blended pellets obtained in step (e) into capsules or sachets.

20. (canceled)

21. (canceled)

22. (canceled)

23. The composition as claimed in claim 1, wherein the extended release multiple unit pellet system composition prepared by coating active pharmaceutical ingredient core spheres with seal coating of 5% buildup using coating polymer selected from polyethylene glycol or hypromellose or Eudragit or Opadry and purified water, functional coating of 50% buildup using ethyl cellulose and/or hypromellose, triethyl citrate, talc, isopropyl alcohol and purified water, second seal coating of 5% buildup over functional coated pellets using coating polymer selected from polyethylene glycol or hypromellose or Eudragit or Opadry and purified water, extragranular portion containing microcrystalline cellulose, copovidone, colloidal silicon dioxide and sodium stearyl fumarate or magnesium stearate for lubricating and compressing to get multiple unit pellet system.

24. The composition as claimed in claim 1, wherein tablet is optionally coated with Opadry ready to use film coating.
